(19) United States
(12) Patent Application Publication
(10) Pub. No.: US 2008/0114173 A1
(43) Pub. Date: May 15, 2008

(54) Crystalline Form of Donepezil Hydrochloride

(76) Inventors: Vijayavitthal Thippannachar Mathad, Hyderabad (IN); Sharat Pandurang Narasapur, Secunderabad (IN); Pravinchandra Jayantila Vankawala, Hyderabad (IN); Manoj Ramesh Khakar, Hyderabad (IN); Ravi rama Chandra Shekar Elati, Hyderabad (IN); Subrahmanyeswar Rao Chalamala, Hyderabad (IN); Arun T., Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/572,949

(22) PCT Filed: Aug. 1, 2005
(86) PCT No.: PCT/US05/27326

§ 371 Date: Jan. 30, 2007

Related U.S. Application Data
(60) Provisional application No. 60/592,516, filed on Jul. 30, 2004.

(51) Int. Cl. C07D 211/32

(52) U.S. Cl. 546/206

(57) ABSTRACT

A process for preparing crystalline form I of donepezil hydrochloride comprises: a) condensing 5,6-dimethoxy-2-piperidin-4-yl-methyl-indan-1-one with benzyl bromide and reacting a condensation product with hydrobromic acid to form donepezil hydrobromide; and b) hydrolyzing donepezil hydrobromide, followed by reacting with aqueous hydrochloric acid.

### INTRODUCTION TO THE INVENTION

The present invention relates to crystalline form I of (±)-2,3-dihydro-5,6-dimethoxy-2-[[1-(phenylmethyl)-4-piperidinyl]methyl]-1H-inden-1-one hydrochloride, or 1-Benzyl-4-[(5,6-dimethoxy-1-indanon)-2-yl]methylpiperidine hydrochloride, having the adopted name “donepezil hydrochloride” and being represented by Formula I.

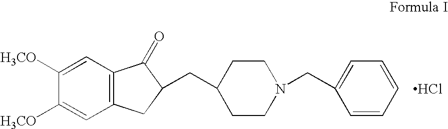

Donepezil hydrochloride is a reversible inhibitor of acetylcholinesterase and is the first agent with this mode of action for the treatment of mild to moderate dementia of Alzheimer's Disease. Products containing donepezil hydrochloride are sold using the trademark ARICEPT.

Many pharmaceutically active compounds have been found to exist in more than one polymorphic form, such as one or more crystalline forms, an amorphous form, and/or sometimes one or more solvated forms. Frequently it is found that the different forms have different physical or chemical properties, such as solubility, hygroscopicity, etc., or have properties that render some form easier to formulate into a pharmaceutical product. In addition, certain forms can have a greater stability than other forms, as shown by a decreased tendency to spontaneously convert into a different polymorphic form.

Unfortunately, predicting the potential number, or even the existence, of polymorphs for a given molecule is not possible. However, regulatory agencies desire that the various polymorphic forms of a compound will be identified before a pharmaceutical product is approved for marketing, because it is essential that a product will remain stable and have predictable properties during its entire shelf life.

Processes for preparing donepezil hydrochloride and some polymorphic forms are found in the following U.S. Pat. Nos. 4,895,841; 5,100,901; 5,606,064; 5,985,864; 6,140,321; 6,245,911; and 6,252,081.

### SUMMARY OF THE INVENTION

In one aspect of the invention, there is provided a process for preparing stable crystalline form I of donepezil hydrochloride comprising:
a) condensing 5,6-dimethoxy-2-piperidin-4-yl-methyl-indan-1-one with benzyl bromide and reacting a condensation product with hydrobromic acid to form donepezil hydrobromide; and
b) hydrolyzing donepezil hydrobromide, followed by reacting with aqueous hydrochloric acid.

In another aspect, the invention provides a donepezil hydrochloride form 1 prepared by the process described above, comprising about 5 to about 6 weight percent water.

In a further aspect, the invention provides a compound 1-Benzyl-4-[(5,6-dimethoxy-1-indanon)-2-yl]methylpiperidine hydrobromide.

### BRIEF DESCRIPTION OF THE DRAWINGS

FIG. 1 is a schematic representation of a process for preparing donepezil hydrochloride.

FIG. 2 is an X-ray powder diffraction pattern of donepezil hydrochloride form I.

FIG. 3 is an infrared absorption spectrum of donepezil hydrochloride form I.

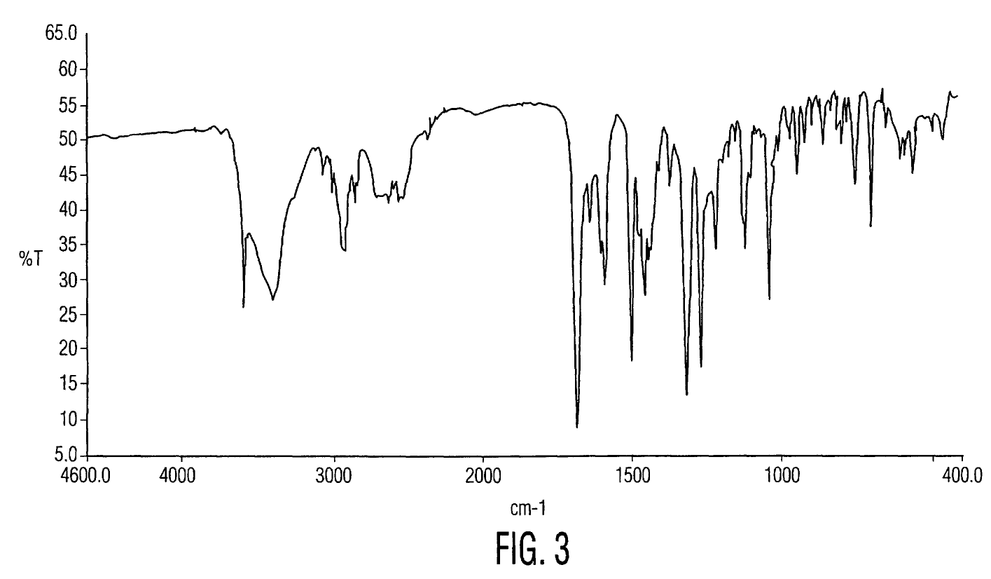

FIG. 4 is a thermogravimetric analysis curve for donepezil hydrochloride form I.

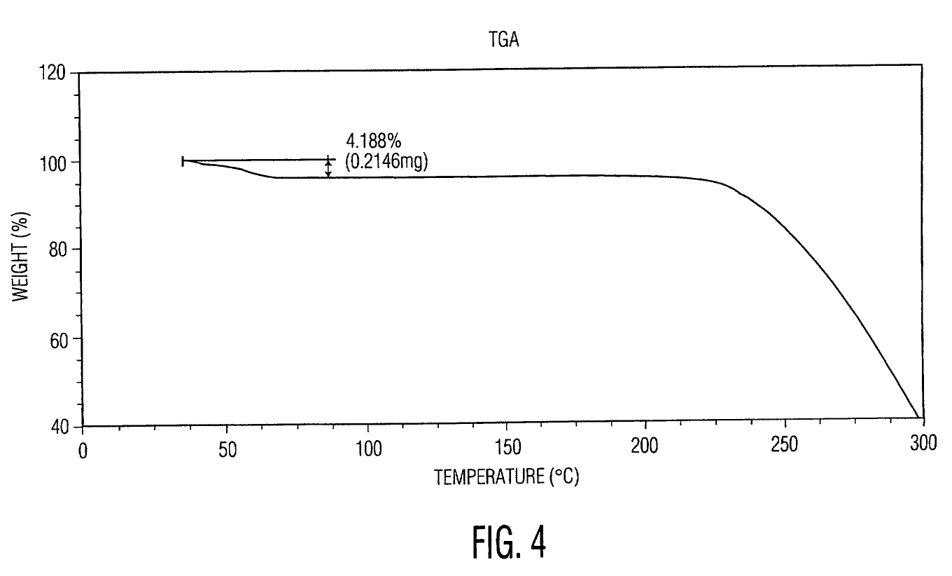

FIG. 5 is an X-ray powder diffraction pattern of donepezil hydrobromide.

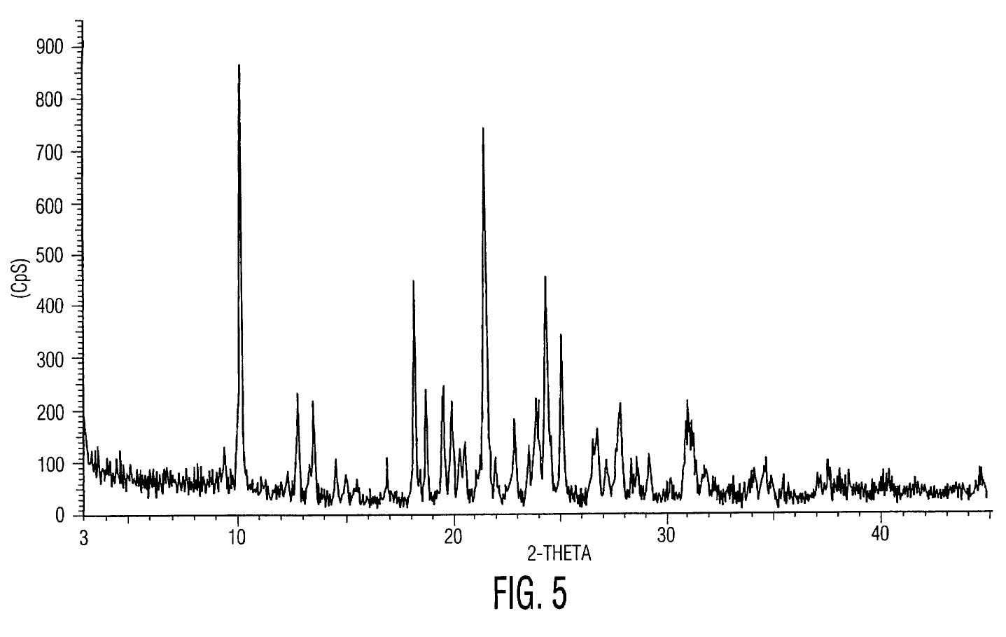

FIG. 6 is an infrared absorption spectrum of 2-(4,4-Dibenzylcyclohexylmethyl)-5,6-dimethoxy-indan-1-one.

FIG. 7 is an infrared absorption spectrum of 1-Benzyl-4-(5,6-dimethoxy-1H-2-indenyl-methyl)piperidine.

### DETAILED DESCRIPTION

A stable donepezil hydrochloride form I contains about 5-6 percent by weight of water. Therefore, it is desired to consistently produce a product having this moisture content. The product that is prepared by the process of this invention is considered to be a monohydrate of donepezil hydrochloride.

The present invention, in one aspect, relates to a process for the preparation of a stable crystalline form I of donepezil hydrochloride comprising the steps of:
a) condensation of 5,6-dimethoxy-2-piperidin-4-yl-methyl-indan-1-one of formula VI with benzyl bromide in presence of a base in a suitable solvent to give donepezil which is subsequently converted in to its hydrobromide salt of formula VI; and
b) conversion of donepezil hydrobromide of formula VI to the free base, and then to the hydrochloride salt of Formula I.

A suitable solvent for conducting the reaction in step a) includes but is not limited to any solvent or mixture of solvents, in which the required components are soluble. Examples include C₁-C₄ straight chain or branched alcohols such as methanol, ethanol, isopropanol and the like; ketones such as acetone, ethylmethyl ketone, diethylketone and the like; ether solvents such as diethyl ether, diisopropyl ether, methyl tertiary butyl ether, tetrahydrofuran and the like; ester solvents such as ethyl acetate, propyl acetate and the like; and acetonitrile; or mixtures of any two or more thereof.

The base that is used in the reaction includes: organic bases, such as but not limited to, C₁-C₁₀ straight or branched chain alkyl amines; and inorganic bases such as hydroxides of alkali metals such as lithium hydroxide, sodium hydroxide, potassium hydroxide, and the like; carbonates of alkali metals such as sodium carbonate, potassium carbonate, and the like; and bicarbonates of alkaline earth metals such as sodium bicarbonate, potassium bicarbonate, and the like.

The donepezil hydrobromide product in step a) is isolated by extraction with a solvent that can be any solvent or mixture of solvents, in which the required component is soluble and is water immiscible. Examples include aromatic hydrocarbons such as benzene, toluene, xylene and the like or their mixtures thereof.

The extraction is typically carried out at the particular pH of the reaction mass, which can range from about 6-10, or about 7-9, or about 7.5-8.2, to make the product free from the process-related impurities.

The product in step a) can be isolated by adding an antisolvent to a solution of the product, useful antisolvents including but not limited to any solvent or mixture of solvents, in which the product is insoluble. Examples include ethers such as diethyl ether, diisopropyl ether, methyl tertiary butyl ether, tetrahydrofuran and the like.

The isolated product of a) is dissolved in chlorinated hydrocarbon solvent such as dichloromethane, ethylene dichloride, chloroform, carbon tetra chloride, and the like, and then is washed with water to make the product free from the starting material of formula VI.

Suitable solvents for conducting the reaction in step b) include but are not limited to any solvent or mixture of solvents, in which the required components are soluble. Examples include aromatic hydrocarbons such as benzene, toluene, xylene and the like, or any mixtures thereof.

The free base that is obtained by the reaction of b) is dissolved in a suitable alcoholic solvent such as C₁-C₄ straight chain or branched alcohols or their mixtures.

The antisolvent used for isolating the product in step b) includes, but is not limited to, any solvent or mixture of solvents, in which the product is insoluble. Examples include ethers such as diethyl ether, diisopropyl ether, methyl tertiary butyl ether, tetrahydrofuran, and the like.

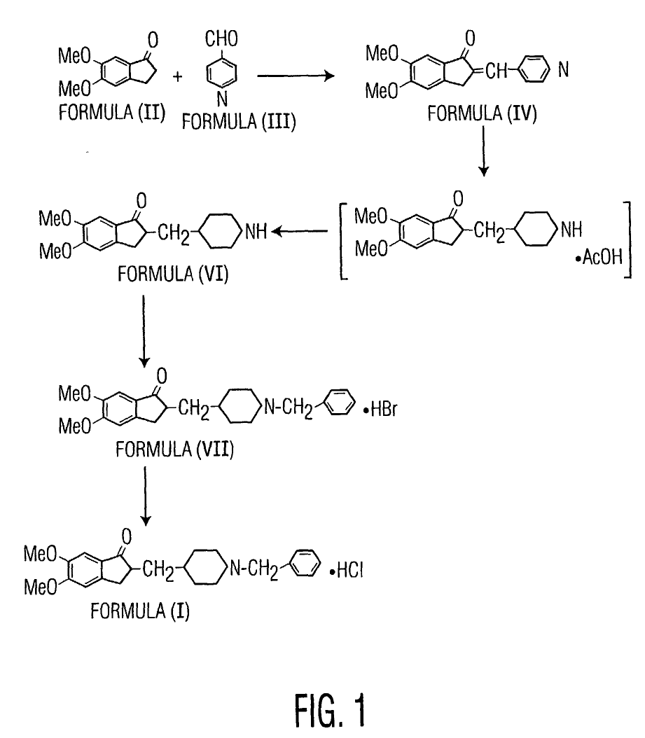

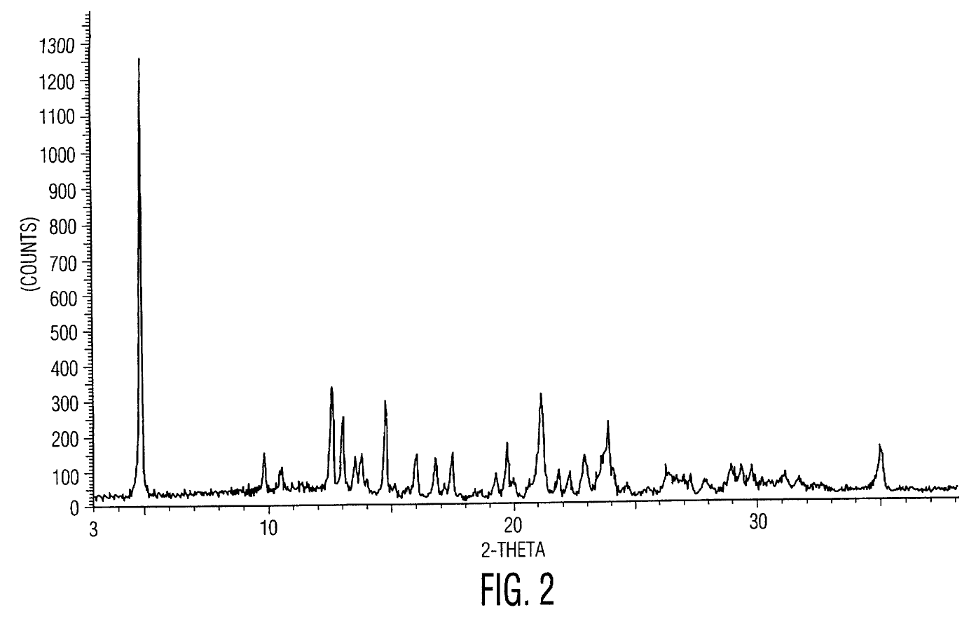

The process for the preparation of crystalline form-I of Donepezil hydrochloride of the present invention is non hazardous and easily scalable. The crystalline form-I of Donepezil hydrochloride prepared by the process of the present invention has been characterized by powder X-ray diffraction pattern analysis as shown in FIG. 2. A schematic representation of the process is depicted in FIG. 1.

A specific embodiment of the process of the invention comprises:
a) combining donepezil hydrobromide, water and toluene at ambient temperature;
b) adding a base to obtain a pH of 12-14;
c) heating the mixture of step b) to a temperature of 45-50° C.;
d) separating the organic layer;
e) extracting the aqueous layer of step c) with toluene;
f) adding sodium hydrosulfite to the organic layer of step (e);
g) separating the organic layer;
h) adding water to the organic layer of step g);
i) separating the organic layer
j) distilling the organic material from step i) to obtain a thick residue;
k) adding methanol to the thick residue of step j);
l) optionally, adding activated carbon to the mixture of step k), and filtering to remove carbon;
m) adding hydrochloric acid to mixture of step k) or step l);
n) adding water to the mixture of step m);
o) cooling the mixture of step (n) to a temperature of 0 to 5° C.;
p) adding chilled methyl tert-butyl ether to the mixture of step (o) at 0-5° C.; and
q) recovering crystalline form-I of donepezil hydrochloride

An embodiment of the invention comprises a process for preparing a highly pure donepezil hydrochloride having low concentrations of the impurities represented by formulas VIII and IX.

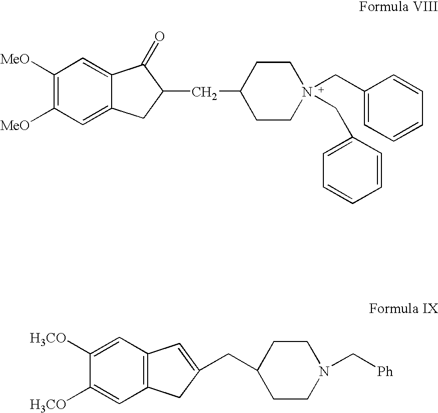

Impurities having Formula VIII and Formula IX will each typically be present at less than 0.1 weight percent of the donepezil hydrochloride, or less than about 0.05 weight percent, or less than about 0.02 weight percent, or less than about 0.01 weight percent.

These impurities are formed during the process for the preparation of donepezil hydrochloride. The selection of suitable solvents for isolation of the product and extraction of the product at pH values as mentioned above with regard to step a) are factors for minimizing these impurities.

The process of the invention is capable of preparing donepezil hydrochloride form I having exceptionally high purity, such as having no more than about 0.1 area-percent of organic compound impurities, as determined by high performance liquid chromatography (“HPLC”). In a particularly pure product of the invention, no individual organic compound impurity will be present in an amount greater than about 0.02 area percent, as determined by HPLC.

A sample of crystalline form I of donepezil hydrochloride is placed into a clear polyethylene bag and closed, and is optionally placed in a black polyethylene bag along with silica gel, and then the bag is placed in a triple laminated bag and the bag is sealed, and the assembly is stored in a HDPE drum. When crystalline form I of donepezil hydrochloride is stored in this manner, there is no significant change to the donepezil hydrochloride over a period of 60 days.

The present invention will be explained in more detail with reference to the following examples, which are provided by way of illustration only and should not be construed as limiting the scope of the invention. In the examples, the provided purity information is for the organic compound content and does not take into account any moisture content.

### EXAMPLE 1

### Preparation of Donepezil Hydrobromide (VII)

5,6-dimethoxy-2-piperidin-4-yl-methyl-indan-1-one (200 g), obtained by the process described in International Publication WO 04/082685, and sodium carbonate (44 g) were suspended in isopropyl alcohol (400 ml) and methanol (1400 ml) and stirred at a temperature of 55 to 60° C. Benzyl bromide (82 ml) was added slowly dropwise, and the resulting reaction mass was stirred at 55-60° C. for 11 hours. The reaction mass was filtered at 25 to 35° C., and water (3000 ml) was added to the filtrate. The compound was extracted from the resulting aqueous solution at a pH of 7.5-8.2 using toluene (1×2000 ml, 4×1000 ml). The resulting toluene layer was washed with water (3×1000 ml) at 65-75° C. and concentrated under vacuum to produce a residue. The residue was dissolved in methanol (800 ml), heated to 60-65° C., and passed through a celite bed. Hydrobromic acid (70.8 ml) was added at 25-35° C. to the resulting filtrate, which was then cooled to 0 to 5° C. for 2-3 hours. Methyl tertiary butyl ether (2000 ml) was added to the cooled filtrate, which was then stirred for 1.5 hours and filtered. The filtered solid was dissolved in chloroform (1400 ml), then the solution was washed with water (3×1000 ml) and concentrated to produce a thick solid. The obtained solid was suspended twice in methanol (2×1000 ml) at 60-65° C. and filtered to afford 174 gm of donepezil hydrobromide. Weight: 174 grams, purity by HPLC: 99.2 percent.

### EXAMPLE 2

### Preparation of Crystalline Form I of Donepezil Hydrochloride

Donepezil hydrobromide (13 Kg), 130 L of water, and 156 L of toluene were added to a reactor and stirred for 10-15 minutes at 25-35° C. A 10 percent caustic lye solution was added to the mixture to adjust its pH to between 12 and 14 and the mixture was then heated to 45-50° C. for 10-15 minutes by using hot water circulation. The reaction solution was allowed to settle at 45-50° C. for 10-15 minutes. The aqueous layer was separated and was charged into a reactor containing 65 L of toluene and stirred at 25-35° C. for 10-15 minutes. After another 10-15 minutes without stirring, the reaction mass was transferred into clean HDPE drums. 5% sodium hydrosulfite solution (65 L) was added to the reaction mass and was stirred for 10-15 minutes at 25-35° C. Then the organic layer was separated. Another portion of the sodium hydrosulfite solution (65 L) was added to the organic layer, with subsequent stirring for 10-15 minutes at 25-35° C. The organic layer was washed two times with 65 L of water at 25-35° C. The organic layer was distilled off under vacuum at temperatures less than 40-45° C. to afford the thick syrup or residue. Methanol (40 L) and activated carbon were added to the residue or syrup and the mixture was heated to 25-35° C. for 10-15 minutes. After the activated carbon was removed by filtration, the filtrate was transferred to a reactor containing methanol (10 L). 10% HCl in methanol (8 L) was added to the reactor over 5-10 minutes at 25-35° C., and then water (13.7 L) was added at 25-35° C. After the reaction mass was cooled to 0-5° C., 150 L of methyl tert-butyl ether (“MTBE”) were added to the reaction mass at 0-5° C., and solids precipitated. The precipitate was separated and washed with a pre-chilled mixture of MTBE and methanol (2:1, v/v). The washed solids were added to pre-chilled MTBE and the mixture was stirred for 10-15 minutes at 0-5° C. The solids were filtered again and washed with pre-chilled MTBE (10 L). The wet material was sifted through a 20 mesh sieve and dried under vacuum to afford crystalline form I of donepezil hydrochloride. Purity by HPLC: 99.9 percent; moisture content: 5.2 percent.

The donepezil hydrochloride form I was milled to produce the particle size distribution shown in the following table, as measured by a Malvern particle size analyzer. In the table, D_{X} means that X volume percent of the particles have a size less than the value given:

| Fraction | Size (μm) |
|---|---|
| D₁₀ | 4.18 |
| D₅₀ | 18.68 |
| D₉₀ | 47.22 |

### EXAMPLE 3

### Preparation of Crystalline Form I of Donepezil Hydrochloride

Donepezil hydrobromide (50 g), water (500 ml), and toluene (600 ml) were placed in a round bottom flask and stirred for 10-15 minutes at ambient temperature. A 10 percent caustic lye solution was added to the reaction mass to adjust its pH to between 12 and 14, and then the reaction mass was heated to 60-65° C. for 10-15 minutes. The aqueous layer was separated and extracted with toluene several times. The combined organic layers were washed with a 5% sodium hydrosulfite solution (2×250 ml) followed by washing with water. The organic layer was distilled to produce a syrup or residue. The syrup or residue was dissolved in methanol (200 ml), carbon was added, and the methanol was refluxed. The refluxed methanol solution was filtered through a celite bed and washed with methanol. Concentrated aqueous HCl (15.8 ml) was added to the filtrate under continuous stirring and the filtrate was then cooled to 0-5° C. Methyl tert-butyl ether (“MTBE”) was added at 0-5° C., and solids precipitated. The solids were filtered and washed with a pre-chilled mixture of MTBE: methanol (2:1, v/v). The washed solids were added to prechilled MTBE, and the mixture was stirred for 10-15 minutes at 0-5° C., then filtered and washed with pre-chilled MTBE. The wet material was dried under vacuum at 45-50° C. to afford crystalline form I of crystalline donepezil hydrochloride.

### EXAMPLE 4

### Process for the Preparation of 2-(4,4-Dibenzyl-cyclohexyl methyl)-5,6-dimethoxy-indan-1-one (VIII)

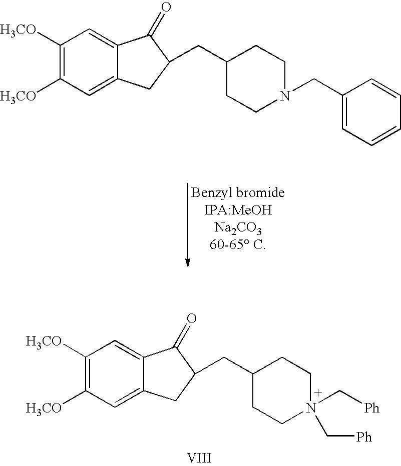

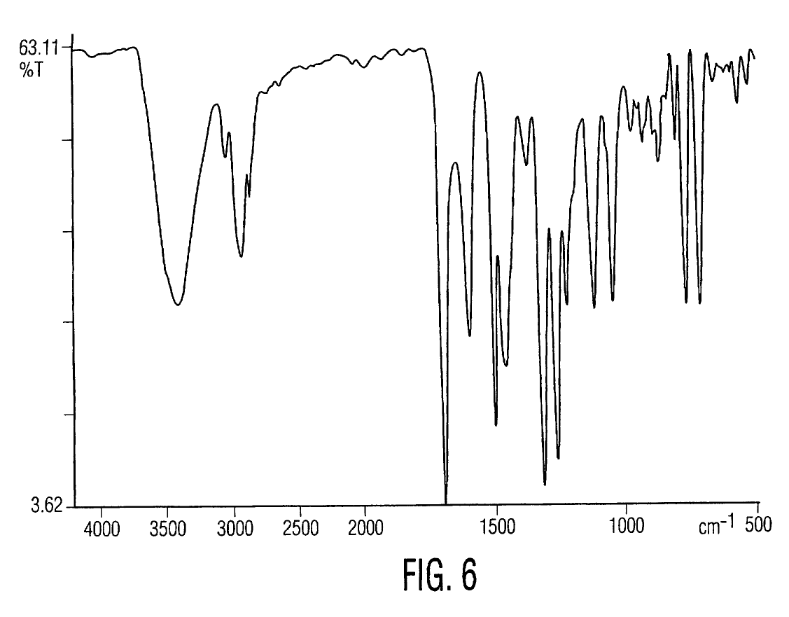

Donepezil (15.0 g, 0.04 moles) was dissolved in a mixture of isopropyl and methyl alcohols (150 ml). To the solution was charged sodium carbonate (8.5 g, 0.08 moles) and the reaction mass was heated to at 60-65° C. Benzyl bromide (8.55 g, 0.05 mole) was added at 60-65° C. The reaction mass was heated to 65-70° C. and maintained for 7-8 hours, while the formation of the dibenzyl compound was monitored by thin layer chromatography. After the reaction was complete, the reaction mass was cooled to 25-35° C. and filtered to isolate the solid 2-(4,4-Dibenzyl-cyclohexylmethyl)-5,6-dimethoxy-indan-1-one (VIII). The Fourier transform infrared absorption spectrum of this compound (in potassium bromide) is shown as FIG. 6.

### EXAMPLE 5

### Preparation of 1-Benzyl-4-(5,6-dimethoxy-1H-2-indenyl-methyl) piperidine (IX)

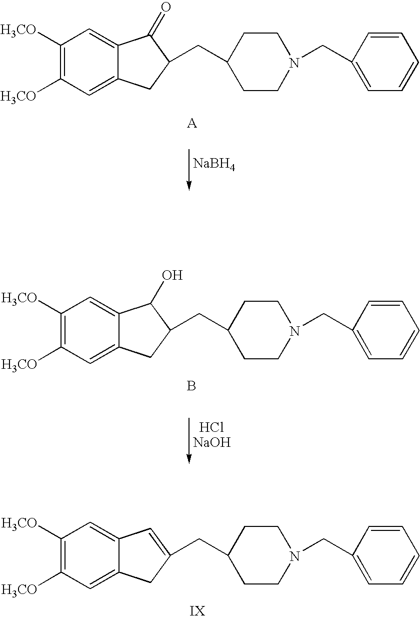

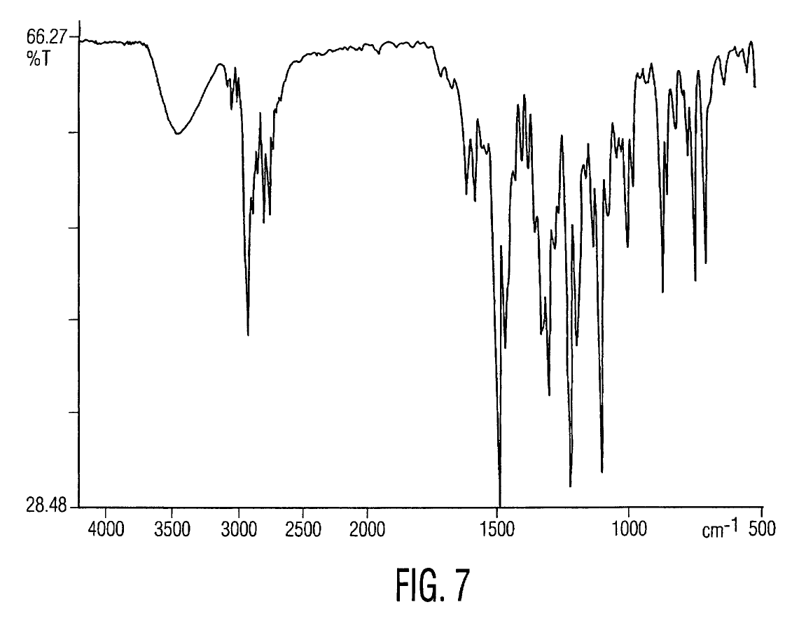

Donepezil (Formula A, 15.0 g, 0.04 moles) was dissolved in toluene (150 ml). To the solution was added sodium borohydride (2.99 g, 0.079 moles) in three portions at 5° C. (maintained by an ice bath). Methanol (75 ml) was added to the reaction mass and stirred at 25-35° C. for 3 hours under a nitrogen atmosphere. The formation of the hydroxy compound of Formula B was monitored by thin layer chromatography. After completion of the formation of the hydroxy compound of Formula B, the reaction mass was quenched with chilled water (150 ml), added slowly over a period of 5-10 minutes. The pH of the resulting reaction mass (mixture of organic and aqueous layers) was adjusted to 0.6 using 10N hydrochloric acid (about 16 ml) and the mixture was stirred at 15-25° C. for 60 minutes. The dehydration of the hydroxy compound of Formula B was monitored by thin layer chromatography and the organic layer was separated. The pH of the aqueous layer was adjusted to 12.6 using caustic lye (about 20 ml) to precipitate the product of Formula IX as an off-white crystalline solid. The solid was filtered and dried at 55° C. to afford 12.9 g of 1-Benzyl-4-(5,6-dimethoxy-1H-2-indenyl-methyl)piperidine (IX). The Fourier transform infrared absorption spectrum of this compound (in potassium bromide) is shown as FIG. 7.

1. A process for preparing stable crystalline form I of donepezil hydrochloride comprising:
a) condensing 5,6-dimethoxy-2-piperidin-4-yl-methyl-indan-1-one with benzyl bromide and reacting a condensation product with hydrobromic acid to form donepezil hydrobromide; and
b) hydrolyzing donepezil hydrobromide, followed by reacting with aqueous hydrochloric acid.

2. The process according to claim 1, further comprising the step of precipitating donepezil hydrobromide by adding an antisolvent for donepezil hydrobromide, before step b).

3. The process according to claim 1, further comprising, in step b), precipitating donepezil hydrochloride by adding an antisolvent for donepezil hydrochloride.

4. Donepezil hydrochloride form 1 that is prepared according to the process of claim 1, having about 5 to about 6 weight percent water.

5. Donepezil hydrochloride form I that is prepared according to the process of claim 1, having no greater than about 0.1 area-percent of total organic compound impurities, as measured by high performance liquid chromatography.

6. Donepezil hydrochloride form I that is prepared according to the process of claim 1, comprising less than about 0.1 area-percent of 2-(4,4-Dibenzyl-cyclohexylmethyl)-5,6-dimethoxy-indan-1-one as an impurity, as measured by high performance liquid chromatography.

7. Donepezil hydrochloride form I that is prepared according to the process of claim 1, comprising less than about 0.05 area-percent of 2-(4,4-Dibenzyl-cyclohexylmethyl)-5,6-dimethoxy-indan-1-one as an impurity, as measured by high performance liquid chromatography.

9. Donepezil hydrochloride form I that is prepared according to the process of claim 1, comprising less than about 0.1 area-percent of 1-Benzyl-4-(5,6-dimethoxy-1H-2-indenyl-methyl)piperidine as an impurity, as measured by high performance liquid chromatography.

10. Donepezil hydrochloride form I that is prepared according to the process of claim 1, comprising less than about 0.05 area-percent of 1-Benzyl-4-(5,6-dimethoxy-1H-2-indenyl-methyl)piperidine as an impurity, as measured by high performance liquid chromatography.

11. Donepezil hydrochloride form I that is prepared by the process of claim 1, comprising less than about 0.02 area-percent of any individual organic compound impurity, as measured by high performance liquid chromatography.

12. A compound 1-Benzyl-4-[(5,6-dimethoxy-1-indanon)-2-yl]methylpiperidine hydrobromide.

13. A process for preparing donepezil hydrochloride form I, comprising adding an antisolvent to a solution comprising donepezil hydrochloride.

14. The process of claim 13, wherein an antisolvent comprises an ether.

15. The process of claim 13, wherein an antisolvent comprises diethyl ether, diisopropyl ether, methyl tertiary butyl ether, or tetrahydrofuran.

16. The process of claim 13, wherein a solution is cooled to about 0-5° C., prior to adding an antisolvent.
